(19) United States
(12) Patent Application Publication
(10) Pub. No.: US 2008/0275310 A1
(43) Pub. Date: Nov. 6, 2008

(54) Apparatus for Monitoring Sweating State by Adjusting Temperature

(76) Inventor: Chang Hwan Kim, Seoul (KR)

Correspondence Address: WEINER & BURT, P.C., 635 N US-23, POB 186, HARRISVILLE, MI, 48740, US

(21) Appl. No.: 11/910,679

(22) PCT Filed: Apr. 6, 2006
(86) PCT No.: PCT/KR06/01269

§ 371 Date: Jun. 24, 2008

(30) Foreign Application Priority Data
Apr. 8, 2005 (KR) 10-2005-0029669

(51) Int. Cl. A61B 5/00

(52) U.S. Cl. 600/300

(57) ABSTRACT

Disclosed is an apparatus for monitoring a sweating state by adjusting skin and core temperatures, in which infrared ray heating means for raising the body temperature and cameras for photographing the sweating states of the upper and the lower part of the body of a person to be diagnosed are provided in a chamber so that the sweating states of the respective parts in accordance with the increase in the body temperature are photographed and are stored in a database and the sweating patterns are analyzed to simply and correctly diagnose the focus and the aching part of the person to be diagnosed. The apparatus includes a chamber in which a person to be diagnosed lies to be diagnosed, a temperature controller provided in the chamber to maintain the temperature of the chamber, body heating means provided in the chamber to emit infrared rays to the body and to raise the body temperature, body temperature sensors for detecting the core and the skin temperature of the body, cameras provided in the chamber to photograph the sweating pattern caused by an increase in the body temperature, a controller for sensing the body temperature of the person to be diagnosed and the temperature of the previously set temperature, and a computer for receiving the sweating pattern of the person to be diagnosed that is transmitted from the cameras to store, mange, and monitor the sweating pattern and for analyzing data on the body temperature of the person to be diagnosed and on the environments of the chamber that are received from the controller to control the controller to maintain the optimal environments. chamber to control the body heating means at a previously set temperature, and a computer for receiving the sweating pattern of the person to be diagnosed that is transmitted from the cameras to store, mange, and monitor the sweating pattern and for analyzing data on the body temperature of the person to be diagnosed and on the environments of the chamber that are received from the controller to control the controller to maintain the optimal environments.

### TECHNICAL FIELD

The present invention relates to a diagnosis apparatus using the sweating pattern of a human body, and more particularly, to an apparatus for monitoring a sweating state by adjusting a temperature by which infrared rays are radiated onto the body of a person to be diagnosed who lies in a chamber to raise the body temperature, to photograph a change in a temperature and a sweating state, and to analyzing the sweating pattern of each part of a human body by a computer so that it is possible to simply diagnose the focus and the aching part of the person to be diagnosed.

### BACKGROUND ART

A digital infrared thermographic imaging apparatus (DITI) for sensing the body temperature of a user to output thermographic images of a focus and normal parts and to diagnose the aching part of a user has been used.

In the DITI, infrared rays are radiated from an infrared ray sensor so that only the wavelength to be sensed by the infrared rays is transmitted by a filter in the infrared ray incident beam source generated from the aching part of a patient to be diagnosed and that the wavelength is incident on a sensing element.

The sensing element converts the incident infrared rays into heat to output thermal image through a color printer using a change in polarization in the sensing material generated by the heat and to thus compare thermal images output before and after curing the patient with each other so that it is possible to diagnose the presence of a disease and the degree of the disease if any.

According to the above-described DITI, a chamber whose temperature can be controlled is provided and infrared ray cameras are provided in the chamber to photograph the entire body of a patient at a distance.

After completing the monitoring, the result is output as a thermal image so that the test content output from an output unit that provides various colors is taken to an examining room and that a doctor makes a diagnosis.

However, the above-method has a disadvantage in that the correctness and repetitiveness of the photographing result deteriorate in accordance with a change in states (environments, meals, and medicines) of a patient.

### DISCLOSURE

### Technical Problem

Accordingly, it is an object of the present invention to provide an apparatus for correctly showing the reaction generated by properly stimulating the sympathetic nervous system in a stable state. Therefore, there is provided an apparatus for monitoring a sweating state by adjusting a temperature according to the present invention, in which a chamber where a person to be diagnosed lies in a state where the sympathetic nerve is stable. And infrared ray heating means for raising the skin and core temperature and digital cameras for photographing the sweating states of the upper part of the body and the lower part of the body of the person to be diagnosed are provided in the chamber to photograph the sweating states of the respective parts in accordance with an increase in the skin and core temperature, to create a database, and to analyze a sweating pattern.

These graphic images are analyzed for the evaluation of the central and peripheral autonomic function.

### Technical Solution

It is another object of the present invention to provide an apparatus for monitoring a sweating state by adjusting the body temperature and the humidity, in which a chamber where the temperature and the humidity are controlled to optimize the stimulus of the sympathetic nerve is provided to minimize the influences that the temperature and the humidity have on the diagnosis and to generate the controlled reaction so that the optimal diagnosis can be performed.

In order to achieve the foregoing and/or other objects of the present invention, there is provided an apparatus for monitoring a sweating state by adjusting the skin and core temperature comprising a chamber in which a person to be diagnosed lies to be diagnosed, a temperature controller provided in the chamber to maintain the temperature of the chamber, body heating means provided in the chamber to emit infrared rays to the body and to raise the body temperature, body temperature sensors for detecting the core and skin temperature of the body, cameras provided in the chamber to photograph the sweating pattern caused by an increase in the body temperature, a controller for sensing the body temperature of the person to be diagnosed and the temperature of the chamber to control the body heating means at a previously set temperature, and a computer for receiving the sweating pattern of the person to be diagnosed that is transmitted from the cameras to store, mange, and monitor the sweating pattern and for analyzing data on the body temperature of the person to be diagnosed and on the environments of the chamber that are received from the controller to control the controller to maintain the optimal environments.

The apparatus further comprises a temperature controller provided in the chamber to maintain the temperature of the chamber in accordance with the control of the controller and a humidity controller provided in the chamber to maintain the humidity of the chamber to be in a predetermined range in accordance with the control of the controller.

The humidity controller comprises a humidity sensor for sensing the humidity of the chamber to output the sensed humidity to the controller and a humidifier that operates in accordance with the control of the controller to supply moisture to the inside of the chamber. The temperature controller comprises a chamber temperature sensor for sensing the temperature of the chamber to output the sensed temperature to the controller and a heater that operates in accordance with the control of the controller to heat the chamber.

The body heating means are infrared ray heating devices provided on the internal top surface of the chamber. The cameras are a plurality of cameras for photographing the sweating states of the upper and the lower part of the body of the person to be diagnosed. The body temperature sensors comprise a core temperature sensor inserted into the mouth of the person to be diagnosed to measure the core temperature of the body and skin temperature sensors attached to the legs, the body, and the face of the person to be diagnosed to measure the skin temperatures of the body.

### DESCRIPTION OF THE DRAWINGS

These and/or other objects and advantages of the invention will become apparent and more readily appreciated from the following description of the preferred embodiments, taken in conjunction with the accompanying drawings of which:

FIGS. 1a and 1b illustrate the outer appearance and the structure of an apparatus for monitoring a sweating state according to an embodiment of the present invention;

FIGS. 2a and 2b are block diagrams illustrating the apparatus for monitoring a sweating state according to the present invention; and

FIG. 3 is a flowchart illustrating the operation processes of the apparatus for monitoring a sweating state according to the present invention.

### BEST MODE

Hereinafter, a preferred embodiment of the present invention will be described with reference to the accompanying drawings.

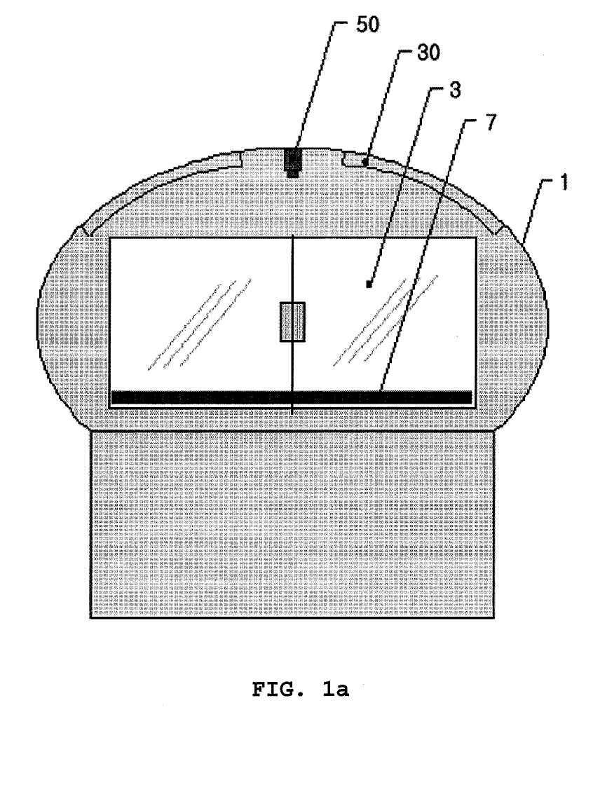

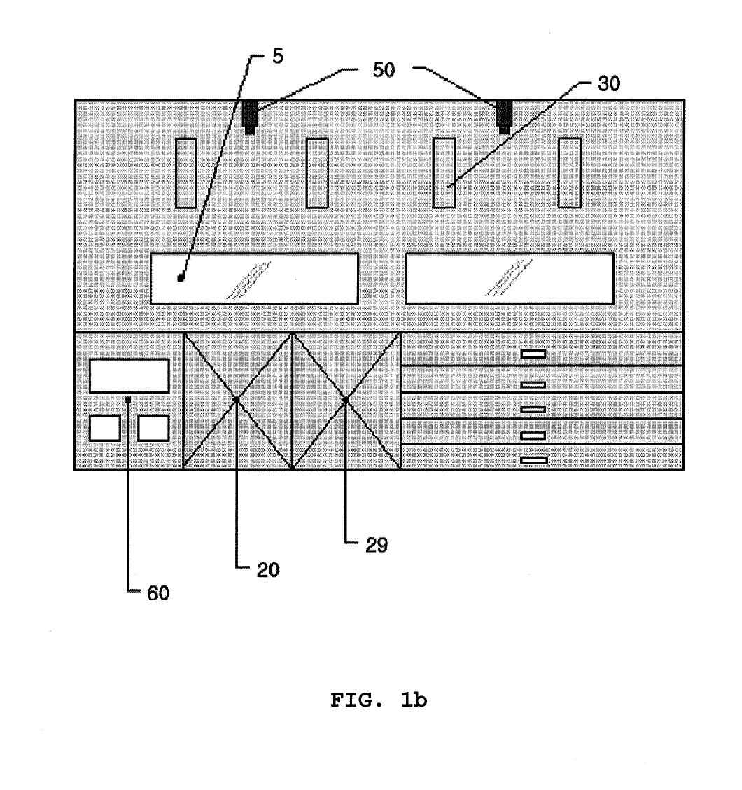

FIGS. 1a and 1b illustrate the outer appearance and the structure of an apparatus for monitoring a sweating state according to an embodiment of the present invention. The apparatus for monitoring a sweating state includes a chamber 1, a temperature controller 10, a humidity controller 20, body heating means 30, cameras 50, and a controller 60.

The chamber 1 includes a space in which a person to be diagnosed lies to be diagnosed. The temperature controller 10 is provided in the chamber 1 to maintain the temperature in the chamber 1. The humidity controller 20 is provided in the chamber 1 to maintain the humidity in the chamber 1 to be in a predetermined range. The body heating means 30 are provided in the chamber 1 to emit infrared rays to a human body to raise the body temperature. The cameras 50 are provided in the chamber 1 to photograph the sweating state caused by raising the body temperature. The controller 60 senses the body temperature of the person to be diagnosed and the temperature and the humidity of the chamber 1 to control the temperature controller 10, the humidity controller 20, and the body heating means 30 in predetermined environments.

As illustrated in FIGS. 1a and 1b, the chamber 1 in which the person to be diagnosed lies to be diagnosed has a size of about 2,000(l)*1,200(w)*2,000(h) [mm] and the mat on which the person to be diagnosed lies is separated from the bottom by a distance of about 600 mm.

A door 3 is preferably provided in the front of the chamber 1 so that the person to be diagnosed enters the chamber 1.

Also, windows 5 that can be opened and closed are provided on the sides of the chamber 1 so that it is possible to observe the person to be diagnosed and to directly provide various sensors in the person to be diagnosed through the windows 5.

At least two cameras 50 are provided on the internal top surface (the ceiling) of the chamber 1 to photograph the sweating states of the upper part of the body and the lower part of the body of the person to be diagnosed.

The body heating means 30 are provided on the internal top surface (the ceiling) of the chamber 1 to emit the infrared rays to the body of the person to be diagnosed.

A humidifier 21, a dehumidifier 29, and the controller 60 are properly arranged under the chamber 1.

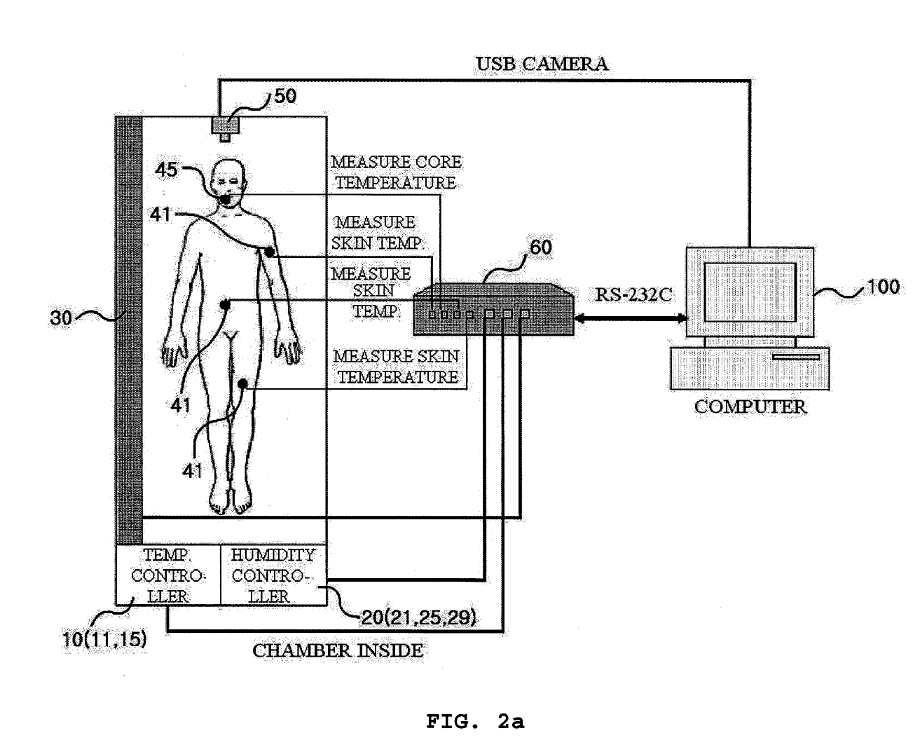

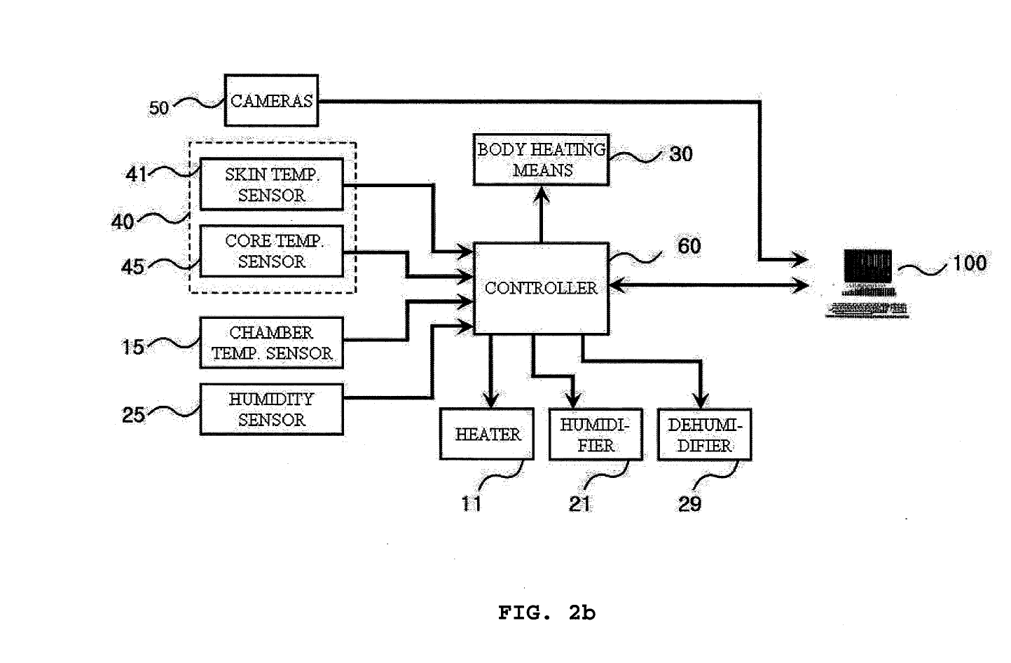

FIGS. 2a and 2b are block diagrams illustrating the apparatus for monitoring a sweating state according to the present invention. The apparatus for monitoring a sweating state includes the temperature controller 10, the humidity controller 20, the body heating means 30, body temperature sensors 40, the cameras 50, the controller 60, and a computer 100.

The temperature controller 10 is provided in the chamber 1 to maintain the temperature of the chamber 1. The humidity controller 20 is provided in the chamber to maintain the humidity of the chamber 1 to be in a predetermined range. The body heating means 30 are provided in the chamber 1 to emit the infrared rays to the body to raise the body temperature. The body temperature sensors 40 are provided (set) in the respective parts of the body to detect the temperatures of the center and the skin of the body. The cameras 50 are provided in the chamber 1 to photograph the sweating state caused by raising the body temperature. The controller 60 senses the body temperature of the person to be diagnosed and the temperature of the chamber 1 to control the temperature 10 and the body heating means 30 at a previously set temperature. The computer 100 receives the sweating pattern of the person to be diagnosed from the cameras 50 to store and process the sweating pattern and analyzes the body temperature of the person to be diagnosed and data on the environments of the chamber 1 that are received from the controller 60 to stably control the controller 60 so that uniform monitoring conditions are maintained.

The humidity controller 20 includes a humidity sensor 25 for sensing the humidity of the chamber 1 to output the sensed humidity to the controller 60 and the humidifier 21 that operates in accordance with the control of the controller 60 to supply moisture to the inside of the chamber 1. The temperature controller 10 includes a chamber temperature sensor 15 for sensing the temperature of the chamber 1 to output the sensed temperature to the controller 60 and a heater 11 that operates in accordance with the control of the controller 60 to raise the temperature of the chamber 1.

The body temperature sensors 40 include a sensor 45 that is inserted into the mouth to measure the core temperature of the body and sensors 41 attached to the legs, the body, and the face to measure the skin temperature of the respective parts.

Although not shown, a speaker and a microphone are preferably provided in the chamber 1 so that the person to be diagnosed and a doctor have a conversation.

When a sweating reaction is measured, the temperature of the chamber 1 that is heated by the temperature controller 10 is preferably maintained to be about 45 to 50° C. and the humidity of the chamber 1 that is controlled by the humidity controller 20 is preferably maintained to be about 35 to 40%.

The body heating means 30 can be controlled so that the skin temperature of the body may be raised to a temperature between 38.5 and 39.5° C. and that the core temperature of the body may be raised to a temperature between 38 and 38.4° C. that is the target temperature in 45 to 65 minutes.

The sensors 40 for measuring the skin and the core temperature may be set in the respective parts of the person to be diagnosed through the windows 5 formed on the sides of the chamber 1 in a state where the person to be diagnosed lies in the chamber 1.

The chamber controlling software recorded in the computer 100 stores the information received from the chamber 1 through an interface such as RS-232C and USB in the computer 100 and analyzes in real time and controls the two cameras 50 provided in the chamber 1 to photograph the person to be diagnosed at predetermined intervals, to store the pictures in a database, and to analyze the sweating pattern of the person to be diagnosed through image processing software.

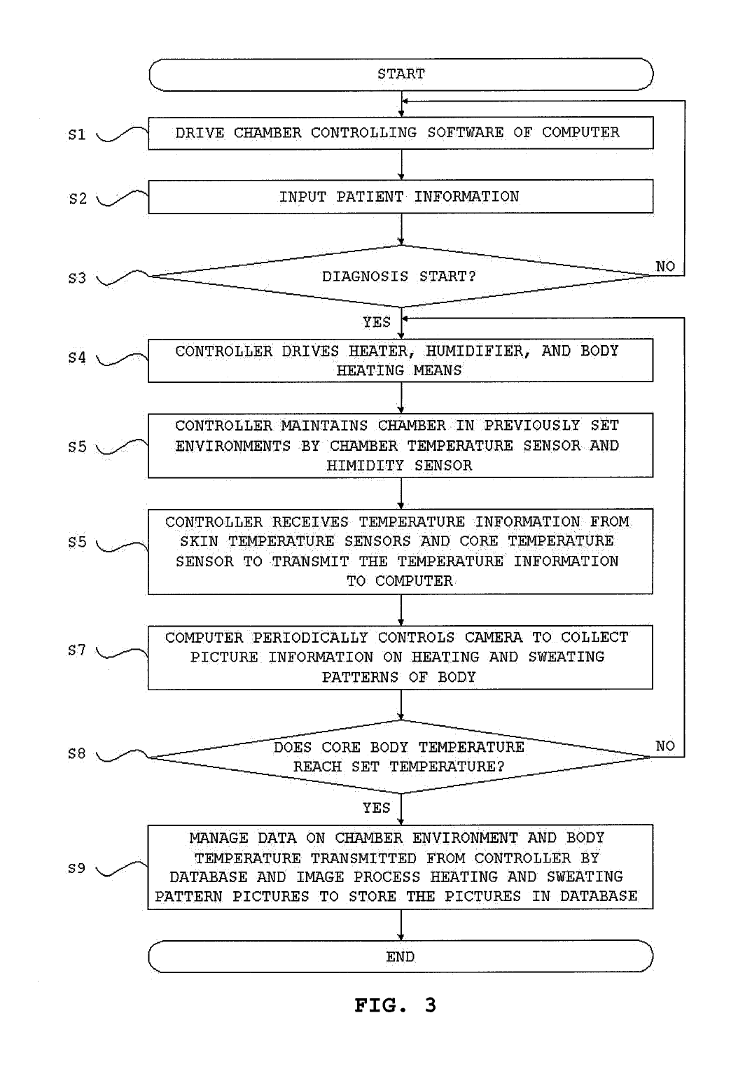

The operation processes of the apparatus for monitoring a sweating state by adjusting a temperature having the above structure will be described with reference to the flowchart of FIG. 3.

After providing the skin temperature sensors 41 and the center temperature sensor 45 in the body of the person to be diagnosed in the chamber 1, the chamber controlling software of the computer 100 is driven to input information on the patient (S1 and S2).

After inputting information on the patient, in a state where communications with various devices (such as cameras and various temperature sensors) in the chamber are initialized, a diagnosis start command is output to the controller (S3).

Therefore, the controller 60 drives the heater 11, the humidifier 21, and the body heating means 30 in accordance with an internal control program to maintain the temperature and the humidity of the chamber 1. The controller 60 receives the sensing signals of the chamber temperature sensor 15 and the humidity sensor 25 to compare the received sensing signals with the previously set temperature and humidity data and to control the heater 11 and the humidifier 21 so that the environments of the chamber 1 are properly maintained. The controller 60 drives the body heating means 30 to emit the infrared rays to the body of the person to be diagnosed so that the skin temperature and the core temperature of the body are raised to a certain temperature (S4 and S5).

In the above-described state, the controller 60 collects data on the skin temperature and the core temperature of the body of the person to be diagnosed that are output from the skin temperature sensors 41 and the core temperature sensor 45 to periodically report the temperature data to the computer 100 (S6).

The computer 100 collects data on the temperature of the chamber 1 transmitted from the controller 60 in real time and the core temperature and the core temperature of the patient to store the collected data in the database and controls the cameras 50 at previously set intervals to receive the sweating patterns of the upper part of the body and the lower part of the body of the person to be diagnosed that are transmitted from the plurality of cameras 50 and to store the sweating patterns in the database (S7).

Then, the computer 100 receives data on the core temperature of the body that is transmitted from the controller 60 to determine whether the core temperature is raised to a previously set temperature and, when it is determined that the core temperature is raised to the previously set temperature between 38 to 38.4° C. (S8), the computer 100 transmits a diagnosis termination command to the controller 60 and image processes the collected pictures of the person to be diagnosed to evaluate the sweating pattern and to store the evaluated sweating pattern in the database (S9).

The controller 60 stops the operations of the body heating means 30, the heater 11, and the humidifier 21 in accordance with the diagnosis termination command of the computer 100.

Although a few embodiments of the present invention have been shown and described, it would be appreciated by those skilled in the art that changes might be made in this embodiment without departing from the principles and spirit of the invention, the scope of which is defined in the claims and their equivalents.

### INDUSTRIAL APPLICABILITY

As described above, according to the present invention, after providing the infrared ray heating means for raising the body temperature and the cameras for photographing the sweating states of the upper part of the body and the lower part of the body of the person to be diagnosed in the chamber, the sweating states of the respective parts in accordance with the increase in the body temperature are photographed and are stored in the database and the sweating patterns are analyzed to simply and correctly diagnose the focus and the aching part of the person to be diagnosed and to perform visual diagnosis through the screen of the computer so that it is possible to improve the reliability of the person to be diagnosed on the diagnosis result.

Also, the environment controller for properly controlling the temperature and the humidity of the chamber is provided to maintain the optimal temperature and humidity of the chamber and to minimize the influences that the temperature and the humidity have on the diagnosis so that it is possible to improve the diagnosis reliability.

1. An apparatus for monitoring a sweating state by adjusting a temperature comprising:
a chamber in which a person to be diagnosed lies to be diagnosed;
body heating means provided in the chamber to emit infrared rays to the body and to raise the body temperature;
body temperature sensors for detecting the core surface and the skin temperature of the body;
cameras provided in the chamber to photograph the sweating pattern caused by an increase in the body temperature;
a controller for sensing the body temperature of the person to be diagnosed and the temperature of the chamber to control the body heating means at a previously set temperature; and
a computer for receiving the sweating pattern of the person to be diagnosed that is transmitted from the cameras to store, mange manage, and monitor the sweating pattern and for analyzing data on the body temperature of the person to be diagnosed and on the environments of the chamber that are received from the controller to control the controller to maintain the optimal environments.

2. The apparatus as claimed in claim 1, further comprising:
a temperature controller provided in the chamber to maintain the temperature of the chamber in accordance with the control of the controller; and
a humidity controller provided in the chamber to maintain the humidity of the chamber to be in a predetermined range in accordance with the control of the controller.

3. The apparatus as claimed in claim 2, wherein the temperature controller comprises:
a chamber temperature sensor for sensing the temperature of the chamber to output the sensed temperature to the controller; and
a heater that operates in accordance with the control of the controller to heat the chamber.

4. The apparatus as claimed in claim 2, wherein the humidity controller comprises:
a humidity sensor for sensing the humidity of the chamber to output the sensed humidity to the controller; and
a humidifier that operates in accordance with the control of the controller to supply moisture to the inside of the chamber.

5. The apparatus as claimed in claim 1, wherein the body heating means are infrared ray heating devices provided on the internal top surface of the chamber

6. The apparatus as claimed in claim 1, wherein the cameras are a plurality of cameras for photographing the sweating states of the upper part of the body and the lower part of the body of the person to be diagnosed.

7. The apparatus as claimed in claim 1, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.

8. The apparatus as claimed in claim 1, wherein a speaker and a mike are provided in the chamber so that the person to be diagnosed and a doctor have a conversation.

9. The apparatus as claimed in claim 1, wherein windows that are opened and closed are provided on the sides of the chamber to attach various body sensors and to observe the state of the person to be diagnosed.

10. The apparatus as claimed in claim 2, wherein the body heating means are infrared ray heating devices provided on the internal top surface of the chamber.

11. The apparatus as claimed in claim 2, wherein the cameras are a plurality of cameras for photographing the sweating states of the upper part of the body and the lower part of the body of the person to be diagnosed.

12. The apparatus as claimed in claim 2, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.

13. The apparatus as claimed in claim 2, wherein a speaker and a mike are provided in the chamber so that the person to be diagnosed and a doctor have a conversation.

14. The apparatus as claimed in claim 2, wherein windows that are opened and closed are provided on the sides of the chamber to attach various body sensors and to observe the state of the person to be diagnosed.

15. The apparatus as claimed in claim 3, wherein the body heating means are infrared ray heating devices provided on the internal top surface of the chamber.

16. The apparatus as claimed in claim 3, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.

17. The apparatus as claimed in claim 4, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.

18. The apparatus as claimed in claim 5, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.

19. The apparatus as claimed in claim 6, wherein the body temperature sensors comprise:
a core temperature sensor inserted into the mouth of the person to be diagnosed to measure the core temperature of the body; and
skin temperature sensors attached to the legs, the body, and the face of the person to be diagnosed to measure the skin temperatures of the body.
